(19) United States
(12) Patent Application Publication
(10) Pub. No.: US 2014/0276657 A1
(43) Pub. Date: Sep. 18, 2014

(54) SYSTEM AND METHOD FOR POST-OPERATIVE LEAK DETECTION

(71) Applicant: THE CLEVELAND CLINIC FOUNDATION, Cleveland, OH (US)

(72) Inventor: Philip Schauer, Hunting Valley, OH (US)

(73) Assignee: THE CLEVELAND CLINIC FOUNDATION, Cleveland, OH (US)

(21) Appl. No.: 14/211,213

(22) Filed: Mar. 14, 2014

Related U.S. Application Data
(60) Provisional application No. 61/788,047, filed on Mar. 15, 2013.

(51) Int. Cl. A61M 27/00

(52) U.S. Cl. CPC A61M 27/00
USPC 604/541

(57) ABSTRACT

The present invention relates a system that can detect a post-operative leak and a related method of use. An amylase detector that includes a sensor can detect a presence of amylase in a fluid secretion from an abdominal organ in a patient's body. The presence of amylase is indicative of the post-operative leak because, while amylase is common in the digestive tract, amylase is not normally found in the abdominal cavity. The amylase detector can alert a monitoring device external to the patient's body of the presence of amylase in the fluid secretion.

### RELATED APPLICATIONS

This application claims the benefit of U.S. Provisional Application No. 61/788,047, filed Mar. 15, 2013, entitled “Post-operative Bariatric Leak Detection.” The entirety of the subject matter of this application is hereby incorporated by reference for all purposes.

### TECHNICAL FIELD

The present invention relates generally to post-operative leak detection and, more specifically, to a system that can detect a post-operative leak based on continuous monitoring of the content of secretions and related methods of use.

### BACKGROUND

A potential complication of bariatric surgery is the development of one or more post-operative leaks. Post-operative leaks developed after bariatric surgery can allow stomach contents to leak into the abdominal cavity, which is a major source of morbidity and mortality due to sepsis, abscesses, nutritional deficiency, and multi-organ failure, and many other complications.

Currently, such post-operative leaks are identified indirectly through patient feedback, radiological studies, or laboratory tests on secretions collected by a surgical drain. However, these indirect methods require a patient to report symptoms and/or a care provider to notice abnormal drainage. In addition to the loss of time to identify the post-operative leaks, the indirect identification methods can also be costly to the patient. A direct identification method can overcome this time delay and allow the care provider to treat the post-operative leaks sooner at a lesser expense to the patient.

### SUMMARY

In one aspect, the present invention includes a system for detecting a post-operative leak. The system includes an implantable post-operative drain device that is configured to drain fluid secretions from a location in the abdominal cavity proximal to an organ corresponding to an operation. An amylase detector that includes a sensor configured to detect amylase within the fluid secretions can be coupled to the drain, comprising a sensor configured to detect amylase within the fluid secretions. The presence of the amylase is indicative of the post-operative leak.

In another aspect, the present invention includes another system for detecting a post-operative leak. The system includes an amylase detector that is configured to be implanted in a patient's abdomen post-operatively along a suture line of an organ. The amylase detector includes a sensor that is configured to detect a presence of amylase that is indicative of the post-operative leak. The system also includes a monitor device that is external to the patient's body and communicatively coupled to the amylase detector. The monitor device is configured to receive an indication of the presence of amylase from the amylase detector and to produce an alert in response to the indication.

In a further aspect, the present invention includes a method for detecting a post-operative leak. The method includes detecting a presence of amylase in a fluid secretion from an abdominal organ in a patient's body. The detection is accomplished by an amylase detector comprising a sensor. Upon the detection, a device external to the patient's body is alerted of the presence of amylase in the fluid secretion. The presence of amylase is indicative of the post-operative leak.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a schematic diagram of an example of a system that can detect a post-operative leak in accordance with an aspect of the present invention;

FIGS. 2, 3, and 4 are schematic diagrams of different potential configurations of the amylase detector in accordance with various aspects of the present invention; and

FIG. 5 is a schematic process flow diagram depicting an example of a method that can detect a post-operative leak in accordance with an aspect of the present invention.

### DETAILED DESCRIPTION

The present invention generally relates to post-operative leak detection. The term “post-operative leak,” as used herein, generally refers to the presence of one or more fluids generally found inside an organ within a patient's abdominal cavity. The term “organ” generally refers to an abdominal organ that contributes to digestion (e.g., the stomach, the small intestine, and/or the large intestine). The term “patient” can refer to any warm-blooded organism including, but not limited to, a human being, a pig, a rat, a mouse, a dog, a cat, a goat, a sheep, a horse, a monkey, an ape, a rabbit, a cow, etc.

The term “post-operative” generally relates to a time after a surgical procedure (e.g., bariatric surgery, colorectal surgery, gastrointestinal surgery, etc.). For example, after abdominal surgery, contents of an organ can leak through a suture. It will be appreciated that leakage (which is in the scope of “post-operative” leakage as used herein) also can occur due to a non-surgical medical condition, and/or due to an accident. In another example, patients with Crohn's Disease and other inflammatory bowel diseases can develop one or more fistulas from a abdominal organ through the abdominal cavity. As another example, a gastric ulcer can rupture and cause stomach contents to leak into the abdominal cavity.

The present invention can detect post-operative leaks based on continuous monitoring of the content of secretions for an indicator of a leak. The indicator of the leak can be a chemical that is present in an organ, but generally not present in the abdominal cavity. Accordingly, the presence of the indicator of a leak within the abdominal cavity indicates the presence of a post-operative leak. The systems and methods of the present invention are described in terms of one such indicator of a leak: amylase (a digestive enzyme that catalyzes the conversion of starch to sugar). Although amylase is described with respect to the systems and methods of the present invention, it will be appreciated that other indicators that are generally present within an organ and not present in the abdominal cavity can be detected in similar ways and correlated to the presence of a post-operative leak.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof. As used herein, the term “and/or” can include any and all combinations of one or more of the associated listed items. Additionally, it will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present invention. The sequence of operations (or acts/steps) is not limited to the order presented in the claims or figures unless specifically indicated otherwise.

In accordance with the present invention, FIG. 1 depicts an example of a system 10 that is configured to detect one or more post-operative leaks. The system 10 can detect the post-operative leaks based on continuous monitoring of the content of secretions for amylase, an indicator of a leak. By monitoring secretions for amylase, the system 10 provides a direct detection method that can detect the post-leak more quickly than traditional indirect detection methods.

The system 10 can include an amylase detector 12 that can provide continuous monitoring of post-operative secretions for the presence of amylase. The amylase detector 12 is communicatively coupled to a monitoring device 16 that can create an alert when amylase is detected by the amylase detector. The term “communicatively coupled” generally refers to the existence of a communicative relationship between one or more components of the amylase detector 12 and one or more components of the monitoring device 16 so that the signal can be transmitted. The amylase detector 12 and the monitoring device 16 can communicate according to a wired connection and/or a wireless connection.

The amylase detector 12 can be configured to be placed in a patient's body (e.g., within an implantable post-operative drain, within a suture line on an organ, etc.). Accordingly, the amylase detector 12 can be constructed from one or more biocompatible materials (e.g., biocompatible materials cause no appreciable immune response when used in a particular application). The monitoring device 16 remains external to the patient's body. In one example, the wired connection between the amylase detector 12 and the monitoring device 16 can extend through a patient's body from the amylase detector 12 within the abdomen to the external monitoring device 16. Accordingly, the wire of the wired connection can be constructed from or coated with one or more biocompatible materials.

In another example, wireless connection between the amylase detector 12 and the monitoring device 16 can be a short-range wireless communication modality that does not produce radiation that is harmful to the patient. Examples of such short-range wireless communication modalities include, but are not limited to: Bluetooth (BT), Bluetooth low energy (BLE), ZigBee, ANT+, and WiFi. The amylase detector 12 can include at least a transceiver to transmit the signal, and the monitoring device 16 can include at least a receiver to receive the signal. The transceiver and the receiver can be configured to transmit and receive the signal according to the same communication protocol.

The amylase detector 12 can be coupled to (or include) a sensor 14 that can detect the presence of amylase within the abdominal cavity (e.g., within a post-operative secretion from a post-operative organ). The sensor 14 can be any type of sensor that can selectively indicate the presence of amylase in the abdominal cavity. For example, detection of amylase within the abdominal cavity by the sensor 14 can complete a circuit within the amylase detector 12 (e.g., the sensor can be a potentiometric sensor, an amperometric sensor, a voltammetric sensor, a galvanic sensor, a magnetic sensor, a capacitive sensor, etc.). In another example, the sensor 14 can undergo a physical transformation (e.g., dissolving at least a portion of the sensor, color change of at least a portion of the sensor, etc.) upon contact with (detection) of the amylase. The detection of the presence of the amylase in the abdominal cavity by the sensor 14 can be indicative of the post-operative leak (e.g., amylase is found within digestive organs, and generally not found within the abdominal cavity).

Upon the detection of the amylase by the sensor 14, the amylase detector 12 can signal the monitoring device 16 of the detection of amylase indicative of the post operative leak. The monitoring device 16 can produce an alert in response to the signal indicating the detection of the post-operative leak. The alert can be any type of alert that is perceivable to a human. For example, the alert can be a visual alert and/or an audio alert.

Although a single amylase detector 12 and a single sensor 14 are illustrated in FIG. 1, it will be appreciated that multiple amylase detectors and/or multiple sensors are within the scope of system 10. For example, the amylase detector can be coupled with (or include) multiple sensors arranged at different locations throughout the abdominal cavity (e.g., at different locations surrounding the post-operative organ). According to another example, multiple amylase detectors and corresponding sensors can be arranged at the different locations throughout the abdominal cavity.

The monitoring device 16 can be a computing device that includes at least a non-transitory memory and a processor. The term “processor,” as used herein includes, but is not limited to, one or more of virtually any number of processor systems or stand-alone processors, such as microprocessors, microcontrollers, central processing units (CPUs), distributed processors, paired processors, and digital signal processors (DSPs), in any combination. The processor may be associated with various other circuits that support operation of the processor, such as random access memory (RAM), read-only memory (ROM), programmable read-only memory (PROM), erasable programmable read-only memory (EPROM), clocks, decoders, memory controllers, or interrupt controllers, etc. These support circuits may be internal or external to the processor or its associated electronic packaging and communicatively coupled to the processor. The non-transitory memory can store computer-executable instructions that can be executed by the processor to facilitate the performance of operations, including functions, actions and/or behaviors. The instructions may be embodied in various forms such as routines, algorithms, modules or programs including separate applications or code from dynamically linked libraries. For example, the computer-readable memory can store instructions to produce the alert upon receiving the indication from the amylase detector, and the instructions can be executed by the processor to produce the alarm.

FIGS. 2, 3 and 4 illustrate examples of different configurations of the amylase detector 12. It will be appreciated that the examples illustrated are not the only potential configurations of the amylase detector. In each case, the position of the amylase detector 12 and the sensor 14 are not intended to be limiting, and are merely examples of possible configurations that can be utilized within system 10. In each case, the amylase detector 12 and corresponding sensor 14 are configured to detect amylase within post-operative fluid secretions.

FIG. 2 shows the amylase detector 12 positioned within (or attached to) an implantable post-operative drain device 22. The implantable post-operative drain device 22 is configured to drain the fluid secretions from a location within the abdominal cavity. The sensor 14 and/or the amylase detector 12 can detect the presence of the amylase in the fluid secretions that is indicative of a post-operative leak. The implantable post-operative drain device 22 can be a shape sufficient to drain the fluid secretions. Additionally, the implantable post-operative drain device 22 can be constructed from one or more biocompatible materials. The implantable post-operative drain device 22 can be positioned in a location near the organ with the fluid secretions without penetrating the organ.

FIG. 3 shows the amylase detector 12 positioned within the collected drained materials (in collection unit 32) from an implantable post-operative drain device 22 that does not penetrate an abdominal organ. The sensor 14 and/or the amylase detector 12 can detect the presence of the amylase in the drained fluid secretions that is indicative of a post-operative leak. The amylase detector 12 can give an indication of the post-operative leak when the amylase reaches the collection unit 32 at end of the implantable post-operative drain device 22.

FIG. 4 shows the amylase detector 12 positioned within the abdominal cavity along a suture line 44 of an organ 42 (e.g., a suture line along a pouch created in the stomach during bariatric surgery) to detect the presence of amylase in fluid secretions from the suture line. The amylase detector 12 and/or the sensor 14 as illustrated in FIG. 3 is constructed of one or more bioresorbable, biodegradable, and/or bioabsorbable materials. As an example, the materials used should be bioresorbable, biodegradable, and/or bioabsorbable within six months.

While aspects of the present invention have been particularly shown and described with reference to the embodiment of system 10, it will be understood by those of ordinary skill in the art that various additional embodiments may be contemplated without departing from the spirit and scope of the present invention. For example, the specific configuration and application of system 10 are merely illustrative; one of ordinary skill in the art could readily determine any number of tools, sequences of steps, or other means/options for placing the above-described apparatus, or components thereof, into positions substantively similar to those shown and described herein. Any of the described structures and components could be integrally formed as a single unitary or monolithic piece or made up of separate sub-components, with either of these formations involving any suitable stock or bespoke components and/or any suitable material or combinations of materials that are should be biocompatible for many applications of the present invention. For some applications, the materials should be bioresorbable, biodegradable, and/or bioabsorbable (e.g., the materials should be chosen so that they exhibit a substantially complete bioresorbtion, biodegrading, or/or bioabsorition within six months).

Though certain components described herein are shown as having specific geometric shapes, all structures of the present invention may have any suitable shapes, sizes, configurations, relative relationships, cross-sectional areas, or any other physical characteristics as desirable for a particular application of the present invention. Any structures or features described with reference to one embodiment or configuration of the present invention could be provided, singly or in combination with other structures or features, to any other embodiment or configuration, as it would be impractical to describe each of the embodiments and configurations discussed herein as having all of the options discussed with respect to all of the other embodiments and configurations.

In view of the foregoing structural and functional features described above, a method in accordance with various aspects of the present invention will be better appreciated with reference to FIG. 5. While, for purposes of simplicity of explanation, the method 50 of FIG. 5 is shown and described as executing serially, it is to be understood and appreciated that the present invention is not limited by the illustrated order, as some aspects could occur in different orders and/or concurrently with other aspects shown and described herein. Moreover, not all illustrated aspects may be required to implement method 50.

FIG. 5 illustrates an example of a method 50 for detecting a post-operative leak (e.g., from an abdominal organ). At 52, a presence of amylase in a fluid secretion from an abdominal organ in a patient's body can be detected (e.g., by sensor 14 coupled to amylase detector 12). The presence of amylase in the fluid secretion is indicative of the post-operative leak. Upon detection of the amylase, a monitor device external to the patient's body (e.g., monitoring device 16) can be alerted (e.g., by the amylase detector 12 across a wired connection or a wireless connection) of the presence of amylase in the fluid secretion.

From the above description, those skilled in the art will perceive improvements, changes and modifications. Such improvements, changes and modifications are within the skill of one in the art and are intended to be covered by the appended claims.

Having described the invention, we claim:

1. A system for detecting a post-operative leak, comprising:
an implantable post-operative drain device configured to drain fluid secretions from a location in the abdominal cavity proximal to an organ corresponding to an operation; and
an amylase detector coupled to the drain, comprising a sensor configured to detect amylase within the fluid secretions,
wherein the presence of the amylase is indicative of the post-operative leak.

2. The system of claim 1, wherein the implantable post-operative drain device is positioned in the location without penetrating the organ.

3. The system of claim 1, wherein the organ is at least one of: the stomach, the small intestine, and the large intestine.

4. The system of claim 1, wherein the amylase detector comprises a wireless transmitter configured to transmit an indication of a presence of amylase within the fluid secretions to a monitor device.

5. The system of claim 1, wherein the amylase detector is coupled to a monitor device by a wired connection and configured to transmit an indication of a presence of amylase within the fluid secretions to the monitor device via the wired connection.

6. The system of claim 1, wherein the sensor is configured to physically transform as an indication of a presence of amylase within the fluid secretions.

7. The system of claim 6, wherein the physical transformation comprises at least one of: a dissolving of at least a portion of the sensor and a color change of at least a portion of the sensor.

8. The system of claim 1, wherein the amylase detector is configured to activate when the detection of the amylase by the sensor completes a circuit of the amylase detector.

9. A system for detecting a post-operative leak, comprising:
an amylase detector configured to be implanted in a patient's abdomen post-operatively along a suture line of an organ,
wherein the amylase detector comprises a sensor configured to detect a presence of amylase indicative of the post-operative leak; and
a monitor device, external to the patient's body and communicatively coupled to the amylase detector, configured to receive an indication of the presence of amylase from the amylase detector and to produce an alert in response to the indication.

10. The system of claim 9, wherein the organ is a stomach and the suture line is along a pouch surgically created in the stomach during bariatric surgery.

11. The system of claim 9, wherein the amylase detector is at least one of bioresorbable, biodegradable, and bioabsorbable within six months.

12. The system of claim 9, wherein the amylase detector and the monitor are communicatively coupled via a wireless connection.

13. The system of claim 9, wherein the amylase detector and the monitor are communicatively coupled via a wired connection.

14. The system of claim 9, wherein a detection of the amylase by the sensor completes a circuit within the amylase detector.

15. The system of claim 9, wherein the detection of the amylase by the sensor causes a physical change to the sensor,
wherein the physical change is at least one of: a dissolving of at least a portion of the sensor and a color change of at least a portion of the sensor.

16. A method for detecting a post-operative leak, comprising:
detecting, by an amylase detector comprising a sensor, a presence of amylase in a fluid secretion from an abdominal organ in a patient's body;
alerting a monitor device external to the patient's body of the presence of amylase in the fluid secretion,
wherein the presence of amylase is indicative of the post-operative leak.

17. The method of claim 16, wherein the amylase detector is coupled to an implantable post-operative drain device,
wherein the implantable post-operative drain device is configured to drain the fluid secretion from a location in the abdominal cavity proximal to the abdominal organ.

18. The method of claim 16, wherein the amylase detector is implanted along suture lines of the abdominal organ,
wherein the amylase detector is at least one of bioresorbable, biodegradable, and bioabsorbable within six months.

19. The method of claim 16, wherein the detection of the presence of amylase at least one of: completes a circuit within the amylase detector and causes a physical change to the sensor.

20. The method of claim 16, wherein the abdominal organ is at least one of: the stomach, the small intestine, and the large intestine.
